(19) United States
(12) Patent Application Publication
(10) Pub. No.: US 2021/0361789 A1
(43) Pub. Date: Nov. 25, 2021

(54) PROCESS FOR PROVIDING ANTIMICROBIAL TREATMENT TO NON-WOVEN FABRICS

(71) Applicant: PSIL HOLDINGS LLOC, Tulsa, OK (US)

(72) Inventors: K. Joy NUNN, Bixby, OK (US); Susan H. BROWN, Tulsa, OK (US)

(21) Appl. No.: 16/319,121

(22) PCT Filed: Jul. 19, 2017
(86) PCT No.: PCT/US2017/042822

§ 371 Date: Jan. 18, 2019

Related U.S. Application Data
(60) Provisional application No. 62/363,946, filed on Jul. 19, 2016.

(51) Int. Cl. A61L 2/10; A61L 2/07; D06M 11/05

(52) U.S. Cl. CPC A61L 2/10; A61L 2202/26; D06M 11/05; A61L 2/07

(57) ABSTRACT

An apparatus and process for disinfecting, and, optionally, sterilizing, fibers and non-woven materials produced from the fibers is disclosed, as well as processes for converting fibers into disinfected and/or sterilized non-woven materials. The process involves contacting the fibers and/or non-woven materials with high temperature steam, and then with UV light, which is preferably UV-C light, or another disinfectant process, such as ozone treatment. The process can also involve process steps such as blending fibers, applying fibers to an air card, subjecting the fibers to one or more carding steps, subjecting the carded fibers to non-woven process steps, and chemically treating the fibers and/or non-woven materials. The resulting non-woven materials can be used, for example, in personal care, baby care (including baby wipes), cosmetic applications, household cleaning, automotive, industrial cleaning applications, industrial uses, and the like.

### BACKGROUND OF INVENTION

Non-woven materials are often prepared in processes that involve consolidating fibers in a web using mechanical bonding, which entangles the fibers to give strength to the web. The two most common methods are needlepunching and spunlacing (hydroentanglement). The industry uses many different terms for spunlaced nonwoven materials, including jet entangled, water entangled, and hydroentangled or hydraulically needled. The term “spunlace” is used more popularly in the nonwoven industry.

Spunlacing uses high-speed jets of water to strike a web so that the fibers knot about one another. As a result, nonwoven fabrics made by this method have specific properties, as soft handle and drapability. Hydroentangled fabrics can incorporate dry-laid webs (carded or air-laid webs as precursors) or wet-laid precursor webs. In some embodiments, a polymer, such as a latex material, is used as a binder for these non-woven materials.

While it is becoming more and more commonplace to use textile waste and other industrial by-products, the technology has generally not developed significantly to manufacture higher quality material from waste. Further, in some embodiments, particularly those where the very young or infirm will be exposed to the products, it can be important to disinfect and/or sterilize the materials, which can be difficult, as the waste materials are exposed to water and other conditions appropriate for microbial contamination at several stages from the time the waste is collected to the time the fibers from the waste materials are converted to non-woven materials.

The present invention provides a process for disinfecting and/or sterilizing non-woven materials, including those made from virgin fibers as well as those made from fibers derived in whole or in part from post-consumer and/or post-industrial waste.

### SUMMARY OF THE INVENTION

Generally, the invention relates to processes for disinfecting, and, optionally, sterilizing, fibers and non-woven materials produced from the fibers. The invention also relates to processes for converting fibers into disinfected and/or sterilized non-woven materials, and for a device suitable for carrying out the process.

The process can start with fibers obtained by deconstructing post-consumer and/or post-industrial waste, such as used textiles and/or leather materials, wood waste, and recycled paper. In one embodiment, the post-consumer and/or post-industrial waste fibers has been cleaned to remove finishes and other chemical treatments, then deconstructed by cutting the textiles and/or leather materials, and forming fibers by using needles to pierce the textiles and/or leather materials and form fibers. Once the waste has been cleaned of finishes/coatings, and cut and resized to form fibers, the fibers approximate virgin fibers. Alternatively, the process can also start with virgin fibers, or blends of virgin and recovered fibers.

Where more than one type of fiber is used, it can be important to have a reasonable homogeneity to the fibers. This can be accomplished, for example, by blending fibers, and, ideally, by intimately blending the fibers.

The blended fibers can then be passed to an airlay card to provide a dry-laid/air-laid web, and to one or more non-woven cards to align the fibers overlying the web with each other, predominantly in the machine direction, then conveyed to a spunlace system.

The spunlace process uses high-pressure water to entangle the fibers. To minimize the possibility of microbial contamination at this stage, the water can be filtered, such as through a filter with pores sized at 0.5 microns or less, to remove bacterial contaminants. The process can include a “dewatering belt” to keep the water concentration relatively low so as to not untangle the purposely tangled fibers.

In some embodiments, rather than using hydroentanglement, the non-woven materials are prepared by bonding fibers using thermal, chemical, or adhesive bonding processes.

Thermal processing can be conducted, for example, by applying a saturant to the fibers, and using a controlled heating process from approximately 100 degrees C. to the cure temperature of the saturant. Typical cure temperatures for saturant formulations range between approximately 150 degrees C. to approximately 190 degrees C. Typical saturants include phenolic resins, acrylics, urea resins, and combinations thereof. Urea-formaldehyde resins are a particular type of saturant.

Thermal bonding can be used to adhere the fibers used to form the non-woven material. In some instances, the strength of the non-woven material can be enhanced by adding a binder, such as a latex emulsion or solution polymer, to provide a chemical bond between the fibers of the substrate.

A chemical dosing system can be used to add chemical treatments to the spunlace non-woven material, and/or add a latex or other polymer to the non-woven material, if desired.

The material can then be dried, calendered, and subjected to a further treatment (referred to herein as a catalytic vapor treatment).

In this treatment, the fibers in the resulting spunlace non-woven materials are opened up by exposure to steam, and then treated with one or more treatment compositions. Within any given treatment station, one or more treatments can be applied. The treatment compositions can be in the form of a vapor, droplets, a stream of liquid, a dispersion, and the like, though vapor can be preferred.

Collectively, the combination of steam and chemical/enzymatic treatment is referred to herein as “catalyzed vapor treatment.” Although the steam is not technically a catalyst, it opens up the fibers and allows them to receive the chemical/enzymatic treatments more efficiently. The water is added in the form of steam, and is removed as the fibers are later isolated, so is not a reactant per se. For this reason, the process has been termed “catalyzed vapor.”

The treatments can serve one or more purposes, such as softening and relaxing twists in the waste materials, providing stain resistance, providing resistance to microbial contamination, providing color, fiber strengthening aids, oils to aid in anti-static formulation, nanotechnology for finishes on fabrics downstream, antimicrobials, such as quaternary ammonium salts, to kill microbial contaminants, and surfactants for personal care products. The treatment compositions in the catalyzed vapor treatment can include one or more of an enzyme, such as a cellulase, protease, lipase, pectinase, or amylase enzyme, a surfactant, which can be a cationic, anionic, zwitterionic, or nonionic surfactant, or a silicone treatment. The selection of the treatment depends on the type of nonwoven materials to be treated.

The first generation of quaternary ammonium salts is known, generically, as benzalkonium chloride or N-alkyl dimethyl benzyl ammonium chloride. The alkyl chain varies in the carbon number, typically from twelve to fourteen carbons, as these tend to be the more powerfully antibacterial sidechains. Second generation quaternary ammonium compounds include ethylbenzyl chloride.

Third generation compounds are typically defined as being mixtures of the first and second generation, i.e., benzalkonium chloride and alkyldimethylbenzylammonium chloride). Their bactericidal action is attributed to the inactivation of enzymes, denaturation of essential proteins and cell membrane rupture. They are usually regarded as a disinfectant in concentrations of 0.25% to 1.6%. The quaternary third generation have an increased biocidal activity detergency and increased bacterial resistance relative to the use of a single molecule.

The fourth generation quaternaries are known as “twin or dual chain quats” or “twin chain” quaternaries,” with chains that are dialkyl linear and without the benzene ring. These include dimethyl ammonium chloride, dioctyl dimethly ammonium chloride, didecyl dimethyl ammonium chloride, and the like. These quaternaries have superior germicidal activity, are low foaming and have a high tolerance to protein loads and hard water.

The fifth generation quaternaries are mixtures of the fourth generation with the second generation, i.e., didecyldimethylammonium chloride, alkyldimethylbenzylammonium chloride, ammonium chloride, alkylbenzyldimethylammonium chloride, and other varieties according to the formulations. The fifth generation quaternary ammonium salts tend to have greater germicidal performance than earlier generations of quaternary ammonium salts.

When the fibers in the spunlace non-woven materials are cotton fibers, the treatment composition can comprise a surfactant or an enzyme, such as a cellulase enzyme. When the fibers are derived from natural hair fabrics, such as cashmere or wool, or a polyester, nylon or polypropylene fiber, the treatment composition can comprise a poly (vinylamine-vinylformamide) copolymer, or other demulsifiers, optionally present in a suitable carrier. These compounds can prevent shrinkage and felting of wool or other natural hair fabrics.

In some embodiments, the fibers are cellulosic fibers, such as wood fibers.

In a further embodiment, the treatment composition comprises an anti-microbial application and/or a biocide.

The treatment processes applied in the various treatment stations can be designed to increase the strength of the fibers, to ensure they are not being weakened, or create additional breakage during the process.

Other treatments can also be applied, including those which increase the strength of the fibers, coloring treatments, disinfecting treatments, and the like.

The treatment or treatments can be applied to the textiles using one or more spray nozzles located above the non-woven materials to be treated or below the porous conveyor. As and after a treatment is applied, whether as a vapor, solution, dispersion, spray, and the like, a vacuum can be pulled below the conveyor, or above the non-woven materials to be treated, respectively, pulling the treatment through the materials to be treated. Excess treatment chemicals can be collected and recycled, if desired.

The treated, moistened non-woven materials can then pass through a decontamination/sterilization station, where they are exposed to one or more of UV light, ethylene oxide, methyl bromide, supercritical or subcritical carbon dioxide, or other decontaminants/sterilants, to remove any microbial contaminants that may be present. Typically, the UV-C radiation has a wavelength between 100 nanometers and 280 nanometers. Exposing the materials (or fibers) to UV-C radiation for sterilization can also modify the surface of the fibers, materials, for example, by increasing wettability and absorbability, and reducing pilling.

In one embodiment, the sterilization step is performed in a sterilization chamber. This can be in the form of a rotating cylinder, with an input end and an output end, wherein the fibers and/or non-woven textile materials traverse from the input end to the output end as they are irradiated by the UV-C radiation source. In one aspect of this embodiment, the rotating cylinder is approximately 1-2 meters in diameter and 3-5 meters long, and is mounted on a stand with an effective slope of −0.12 to −0.16. It can also include a highly reflective interior surface.

The UV-C radiation source typically delivers UV-C radiation with a wavelength between 100 nanometers and 280 nanometers. In one embodiment, the UV-C radiation source is a pulsating UV-C light source, which may consume relatively less energy during operation than other UV light sources. Alternatively, the UV-C radiation source can be a constant UVC light source.

The interior surface of the rotating cylinder can be disposed with a plurality of rows of opening slats which are angled with a travel axis along which the non-woven materials travel in the rotating cylinder, and the rows can be offset from each other. The opening slats can be made of a highly reflective material.

From there, the non-woven materials can be wound up, if desired, and stored for later use to produce finished products, such as non-wovens for use in personal care, baby care (including baby wipes), cosmetic applications, household cleaning, automotive, industrial cleaning applications, industrial uses, and the like.

The present invention will be better understood with respect to the following Detailed Description.

### BRIEF DESCRIPTION OF THE DRAWINGS

FIG. 1 is a flow chart showing one example of how the processes described herein can be carried out.

FIG. 2 is a chart showing the effectiveness of UV exposure as a disinfecting treatment.

FIG. 3 is a top view of a representative UV chamber that can be used to treat the fibers/non-woven materials described herein, showing how a conveyor belt traversing a UV chamber.

FIG. 4 is a front view of representative UV chamber, showing a conveyor belt traversing the chamber, under a series of UV lamps.

FIG. 5 is a side view of representative UV chamber, showing a conveyor belt traversing the chamber, under a series of UV lamps.

FIG. 6 is a chart showing the vapor pressure/temperature characteristics of water, saturated steam, and superheated steam, in terms of temperature (° C.) and pressure (mPa).

### DETAILED DESCRIPTION

Generally, the invention relates to processes for providing spunlaced non-woven materials that are treated to remove microbial contaminants.

The non-woven materials include one or more natural or synthetic fibers, or blends thereof. These fibers can be derived from virgin materials, and/or from post-consumer and/or post-industrial waste or a combination or virgin and post-industrial or post-consumer fibrous materials.

Natural fibers include those derived from plant-based materials, such as jute, sisal, hemp, and cotton, animal hair or protein-based fibers, such as wool, silk, mohair, cashmere, camel hair, natural silks, ramie, coir, and the like, and leather. Fibers can also be obtained, for example, from bamboo, wood, eucalyptus, coconuts, and bananas. Leather waste is typically obtained from shavings through cutting and assembling processes, and from post-consumer or post-industrial waste.

Synthetic fibers include those produced from polymers based on hydrogen, carbon, nitrogen, and oxygen. Nylon, polyester, acrylic and polyolefin fibers account for more than 90 percent of synthetic fiber production. Representative synthetic fibers include polyester fibers, polyolefin fibers, such as polypropylene fibers, and polyaramid fibers.

The present invention will be better understood with reference to the following definitions.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, an airlay process is a nonwoven web forming process that disperses fibers into a fast moving air stream and condenses them onto a moving screen by means of pressure or vacuum.

As used herein, a calender is a machine used to bond sheets of fabric or film to each other or to create surface features on these sheets. It consists essentially of two or more heavy cylinders that impart heat and/or pressure to the sheets that are passed between them. The rollers can be mirror-smooth, embossed with a pattern, or porous. Calendering is a mechanical finishing process used to laminate and to produce special surface features such as high luster, glazing and embossed patterns.

As used herein, a card is a machine designed to separate fibers from impurities, align and deliver them to be laid down as a web or to be further separated and fed to an air laid process. The fibers in the web are aligned with each other predominantly in the machine direction. The machine consists of a series of rolls and drums that are covered with many projecting wires or metal teeth.

As used herein, the spunlace process can be defined as a nonwovens manufacturing system that employs jets of water to entangle fibers and thereby provide fabric integrity. Softness, drape, conformability, and relatively high strength are the major characteristics that make spunlace nonwoven unique among nonwovens.

As used herein, cleaning is the process of substantially removing unwanted surface residues, including solid, liquid, gaseous, organic and inorganic, particulate, radiological and biological contaminants. Preferred cleaning methods typically use a minimal amount of chemical and physical energy in an optimum combination to remove unwanted surface residues.

As used herein, disinfecting is the destruction of all vegetative microorganisms, mycobacteria, small or non-lipid viruses, medium or lipid viruses, fungal spores, and some but not all bacterial spores. Preferred disinfecting methods typically use a minimal amount of chemical and physical energy in an optimum combination to remove or deactivate disease or infection causing microbes.

As used herein, sterilization is defined as a process that will destroy all forms of microbial life on an article or substrate. In practice, in order for a product to be labeled “sterile”, the product is treated with a process which has been validated to produce a SAL of 10-6. The SAL (sterilization assurance level) for a process is a measure of the percent reduction or number of logarithmic reductions (D values) brought about by the sterilization process. For example, a sterilized article having a SAL of 10-6 has a probability of contamination of less than about 1 in one million. Thus, sterilization of a medical device is the process of inactivating trace microbial contaminations not chemically or physically removed by rigorous pre-cleaning and disinfection processes.

### I. Obtaining Clean Fibers for Use in Non-Woven Materials

In some embodiments, virgin fibers are used, in other embodiments, fibers derived from post-consumer and/or post-industrial textile and/or leather materials are used, and in still other embodiments, blends of post-consumer/post-industrial and virgin fibers are used.

Examples of waste materials include:

a) non-conforming materials, defined as materials that do not meet their intended product use,

b) post-industrial textile waste, including slitter waste, pattern, cutting, and trim waste, yarn waste, ginning and carding waste, remnant, and numerous other stages of processing,

c) post-consumer waste, including various types of used product waste unsuitable for reuse in its current state. Examples of post-consumer waste include textiles, flooring (rugs and carpets), wood, leather, and the like.

Where fibers are derived, in whole or in part, from post-industrial and/or post-consumer sources, such as textiles and leather materials obtained from landfills, it can be important to remove surface finishes and/or chemical treatments applied to the materials before seeking to deconstruct the materials into fibers. These finishes/treatments can typically be removed using enzymes, solvents, and the like. This is referred to herein as a “cleaning” process.

In one embodiment, a plurality of units is used to treat the textiles or leather to remove these finishes/treatments. Each unit can be flushed with an aqueous fluid, which causes organic polymeric coatings and treatments, and any organic solvents used to remove them, to rise above the top surface of the aqueous fluid. The organic coatings, treatments, and/or solvents can then be removed, for example, by suction, decantation, by draining from appropriately placed ports, or other means known to the art.

The water can be drained. If desired, the “cleansed” leather and aqueous fluid can be passed through a centrifuge equipped with a centrifuge bag, which allows water to pass through, and retains the leather.

Once the surfaces/coatings have been removed, the textiles/leather can be dried, for example, to a moisture content approximating that of textiles stored at room temperature in an ordinary climate, i.e., to a moisture content of between about 6 and 10 percent by weight.

The resulting “cleansed” textiles/leather can now be positioned on a conveyor, such as a stainless steel grate conveyor, and transported to one or more cutting stations.

Typically, waste textile materials are obtained in a variety of different sizes, and can be initially reduced to a relatively common size to facilitate further conversion to a fiber-based material. In one embodiment, scraps of waste textile material are reduced in size in two or more separate stages.

In a first stage, it can be useful to cut the materials to a more uniform size for further treatment, for example, using a rotary knife. In this stage, the scraps can be cut, for example, to a size in the range of between about 0.5 and about 3 inches in length and in width, and are generally square or rectangular in shape.

These more uniform pieces of textiles/leather materials can be subjected to further cutting steps to produce fibers. For example, the next step can involve size reduction, the materials are cut into relatively shorter sections.

In one embodiment, the scraps are reduced in size in two separate stages. Material size reduction in the initial stage can be performed by a guillotine cutter, and all subsequent fibers produced from this action which are less than 3 mm long can be filtered out of the process. The segregated fibers which are less than 3 mm long can then be moved to a secondary process where they are used in an end-use application appropriate to their size.

A secondary fiber reduction can occur by passing the materials through an enclosed tunnel equipped with a series or rotary knives. In another embodiment, the materials can be passed through pairs of cylinders with a coat of wire or small pins.

The paired cylinders rotate inwardly in a manner that combs or extracts the fibers. In a third embodiment, the materials can be passed under or through cylindrical cutting heads with spiral cutting edges. The edges of the cutting instrument have pointed projections along the spiral ridges which also acts in a combing and extraction method of the fibers. The resulting fiber can then be further refined, if necessary, through the rotary cutting blades allowing for even more accurate fiber length processing.

The focus of this type of fiber reduction operation is to return fibers to the process which measure between 3 mm and 9 mm in length, dependent on the downstream application requirements. In one embodiment, fewer than 5% of total fibers are less than 3 mm long and fewer than 3% of fibers are longer than 9 mm, with the optimum fiber length necessary for a quality non-woven products measuring from 6 mm to 7 mm.

### II. Intimate Blending

Where more than one type of fiber is used to prepare the non-woven material, it can be important to intimately blend the fibers to form a uniform, homogeneous fiber mixture. Ideally, any such blending is carried out such that the composition of the blend is fairly uniform, i.e., the composition varies by no more than 5%, preferably no more than 20%, at any position in the blending box.

The fibers can be physically moved from the cutting/comminution stations, using various means, such as carts, fork trucks, lifts, and the like, so that they are positioned over one or more blending boxes. A representative size for a blending box is approximately 10 feet wide and 20 feet long, but bigger or smaller boxes can be used depending on the volume of production required.

One way to do this is to use a blending box. Fibers can be introduced into blending boxes, for example, using negative pressure, for example, gravity. In one aspect of this embodiment, the materials are moved through duct work using air pressure, where a change in air pressure in a desired location allows the material to drop into the blending box.

### III. Air-Carding to Form a Web or Application to an Existing Web

The blended fibers can then be conveyed to an area where they are air-carded to form a web, or applied to an existing air-carded web.

### IV. Non-Woven Carding

The blended fibers, after being placed on the web, can be subjected to one or more carding operations to align the fibers in a unified direction to form a web or multiple webs to maintain an optimum weight for the targeted product.

### V. Spunlace and Other Non-Woven Processes

In the preferred embodiment, the blended fibers, are placed into a single or series of non-woven card/s to form a lightweight web and subjected to and then be subjected to a spunlace, or hydroentanglement, process. In this process, a jet of water bombards the fibers, physically entangling them.

Because water can include microbial contamination, it is advantageous to use water that has been subjected to a filtration step to remove microbial contaminants. One such type of filter has pore sizes less than about 0.5 microns, more preferably, less than about 2 microns.

Before, as, or after the fibers have been entangled to form a web, they can be treated with one or more chemicals, applied, for example, from a chemical dosing station.

Ideally, the spunlace system has a dewatering belt, so that water leaves the system and is therefore not present to detangle the hydroentangled fibers.

This process, as well as other non-woven processes, are described in more detail below.

In one aspect of this embodiment, a fiber furnish including the fibers described herein is processed using known nonwoven manufacturing processes to produce an intermediate web. For example, one can form a mat with a density of 10-220 gram/m².

Since nonwoven fabrics are prepared from a fibrous web, the characteristics of the web significantly affect the physical properties of the final nonwoven product. These characteristics are derived from the web geometry, which is typically a function of the process by which the web is formed. Web geometry includes characteristics such as the orientation of the fibers, whether the fibers are oriented in a predominate direction or whether their orientation is random, the shape of the fiber (straight, hooked or curled), the extent of fiber entanglement, and fiber compaction. The characteristics of the nonwoven web are also a function of the fiber length (and uniformity), diameter uniformity and web weight.

The decision as to which process is employed for web formation was conventionally determined by the length of the fiber. Historically, the methods for forming of nonwoven webs from virgin uniform textile length fibers was performed using a drylaid (carding) process or by an airlaid process, while nonwoven web formation using short uniform length fibers performed using a wetlaid process on paper making equipment.

It is contemplated a composite nonwoven web can be prepared by any known nonwoven process, such as airlaid or wetlaid processes. However, due to the abundant availability of paper making machinery, particularly in the United States, it can be preferred that an intermediate web be formed using a wetlaid process on a conventional paper making machine. It should be understood, however, that an intermediate web can alternatively be formed using a drylaid and particularly airlaid process.

In another embodiment, after the fibers are refined and the fiber furnish formed, the refined fibers are transported to the headbox of a conventional paper making machine, such as a conventional fourdrinier machine, where they can be fed continuously onto a wire, thus forming the intermediate web.

Typically, nonwoven webs produced on a conventional paper making machine range in basis weight from 201b.-1501b./3000 ft² (approx. 32-244 g/m²). The composite web which is produced by the present process can far exceeds this range. Basis weight composite webs in excess of 400 lb, and commonly in the 600 lb-900 lb/3000 ft. (approx. 975-1500 g/m) range, are contemplated using the present process.

In general, synthetic fibers are stronger, more uniform, more flexible, and less compatible with water than natural fibers. Due to their flexibility and strength, synthetic fibers frequently entangle (flocculate) when they are dispersed in water. Due to their propensity to cause flocculation, synthetic fibers have limited their use in nonwoven webs, particularly when processed with wetlaid processes on conventional paper making equipment.

The conventional strategy for reducing flocculation of fiber due to the presence of synthetic fibers is to increase the dilution by adding additional water to the fiber furnish. While the addition of lots of water operates to physically separate the synthetic fibers in the fiber furnish, the volume of water required necessitates specialized equipment. The circulation of this volume of water also significantly increases the expense of production of the nonwoven web. In addition, the volume of water must be drained through the wire of the paper making machine without interrupting the formation of the web.

An alternative, preferred, solution to an increase in the volume of water in the fiber furnish is to add a polymeric surfactant. Polymeric surfactants attach to the surfaces of fibers at their interface with water when the fibers are suspended in water.

On a molecular level, polymeric surfactant molecules include multiples of both a hydrophilic segment and a hydrophobic segment. Since synthetic fibers are hydrophobic by nature, the hydrophobic segment of the molecules bonds with the synthetic fiber while the hydrophilic segment of the molecule bonds with the surrounding water. The result is that an area of higher viscosity is created around the surface of the synthetic fiber with only a slight increase in the viscosity of the suspension water. Thus, passage of water through the wire in a wetlaid web formation is not significantly affected. The areas of higher viscosity created around the synthetic fibers act as a lubricant which allows adjacent fibers to slide past each other in suspension in the fiber furnish without entanglement. As a result, flocculation of the synthetic fibers is greatly reduced, if not eliminated.

A polymeric surfactant that is suitable for this purpose includes relatively low (10,000 to 200,000) molecular weight ethylene oxide based urethane block copolymers. Commercial formulations of these polymeric surfactants are available commercially from Rohm and Haas under the trademarks Acrysol RM-825, Acrysol RM-8W, and Acrysol Rheology Modifier QR-108, QR-375 and QR-1001.

The polymeric surfactant is preferably added to the water suspension prior to introducing the fiber component, most preferably before the fibers are blended with one or more particles as described herein to form the furnish to be delivered to the wire of a conventional paper making machine. By a combination of draining and/or pressing, water is removed from the intermediate web until it includes approximately 50% by weight water and 50% by weight fiber solids. The intermediate web is then conveyed to a binder station where the composite web is saturated with a binding agent.

The binding agent typically comprises approximately 3%-30% of the composite web, when dried. Therefore, the properties of the binding agent are selected and directly affect the characteristics of the composite web. By adding the relatively smaller particles and/or relatively shorter fibers described herein, the amount of binding agent needed to produce the webs can be reduced.

Binding agents used to form the composite web are preferably of the type which are capable of binding the fiber and particle components to one another. Most preferably these binding agents comprise organic polymer materials which may be heat fused or heat cured at elevated temperatures to bind (bond) the fibers and to provide desired characteristics, such as hydrophobicity, moldability, or stability to consumer and/or industrial products formed from the composite web.

Suitable binding agents include polymeric materials in the form of water dispersed emulsions or solutions and solvent based solutions. These polymer emulsions are typically referred to as “latexes.” With regard to the present invention, the term “latex” refers very broadly to any aqueous emulsion of a polymeric material.

Commercially available latexes have been optimized to promote adhesion to hydrophobic synthetic fibers which may be available in the scrap fiber component of the present process. The range of chemical modifications to latexes which are commercially available is large and designed to meet almost any desired characteristic of the composite web or end use requirement of products manufactured therefrom.

Latex materials used as binding agents in accordance with the present process can range from hard rigid types to those which are soft and pliable (rubbery). Moreover, these latexes may be either thermoplastic or thermosetting in nature. In the case of thermoplastic latex, the latex may or may not be a material which remains permanently thermoplastic. The latex binding agents used in the present process may include non-crosslinked latex, which is preferred. Alternatively, such binding agents may be of a type which is partially or fully cross-linkable, with or without an external catalyst, into a thermosetting type binder. Listed below are several examples of suitable binding agents for use with the present process. It should be understood that the present invention is not limited to the specific examples listed in the categories defined below as other suitable binding agents are contemplated depending upon the desired characteristics in the composite web. Suitable thermoplastic latex binders can be made of one or more of the polymeric materials described herein, specifically, one or more of polyvinyl alcohol, polyethylene vinyl alcohol, polyvinyl acetate, polyvinyl alcohol, acrylic polymers, polyvinyl acetate acrylate, polyacrylates, polyvinyl acetate, polyethylene vinyl acetate, polyethylene vinyl chloride, polyvinyl chloride, neoprene, polystyrene, polystyrene acrylate, polystyrene/butadiene, polystyrene/acrylonitrile, polybutadiene, polybutadiene/acrylonitrile, polyacrylonitrile/butadiene/styrene (ABS), polyethylene acrylic acid, polyethylene urethanes, polycarbonate, polyphenylene oxide, polypropylene, polyesters, and polyamides.

The latex can be altered by carboxylation, or by adding reactive groups, to enhance the physical and chemical properties of the latex. The properties can also be improved by compounding with chemical modifiers, such as thickeners and protective colloids; surfactants to improve stability, wetting and penetration; water-miscible organic liquids added as temporary plasticizers, defoaming agents, or humectants; and water soluble salts, acids, and bases added to adjust pH, alter flow properties, and stabilize the latex polymer against heat and light breakdown.

In some embodiments, a thermoset binding agent can be used. Representative thermoset binding agents include epoxy, phenolic, bismaleimide, polyimide, melamine, melamine/formaldehyde, polyester, urethane, and urea/formaldehyde resins.

The binding agent can be added to the intermediate web using wet saturation or dry saturation. In the wet saturation process, the intermediate web is pressed to about 50% solids and then the saturation fluid containing the binder is added to the web. As the web passes through the wet saturation section, it imbibes the fluid. Finally, the web passes through a second press which removes water and a portion of the binder. The web then enters a dryer section where the remaining water is removed. In the dry saturation process, the formed web is pressed to about 50% solids and then it enters a dryer section where the remaining water is removed. The dry web is then wetted with a fluid containing the binding agent. The fluid can be added by roll coating, bath, dip and squeeze, sprayer, or curtain application methods.

For certain binder compositions, it may be advantageous to convert the fluid containing the binding agent into a foam. After a period of time to allow for penetration, the web passes into a dryer section where water is removed.

In the preferred embodiment of the present process which is wet saturation, the binding agent displaces the water in the intermediate web. In this way, the intermediate web becomes saturated with binding agent.

Optionally, additional materials can be added to the binding agent to produce a desired characteristic in the composite web, and thereafter the consumer and/or industrial product formed therefrom. These materials could vary from pigments to provide color, odor adsorbents or materials to provide a fragrance, fire retardant materials and the like. Such optional additives are described elsewhere herein.

As stated, it should be understood that the materials which may be added are not limited to narrow categories. Furthermore, one or more of the above may be added as required. “When added, multiple types may be adhered to the same fibers in the composite web. Also, the specific examples listed in the categories identified above are by no means exhaustive nor are the categories intended to be limiting for the purpose of the present invention.

Once the intermediate web is saturated with binder, it may be further pressed to remove excess binding agent. Pressing the web is preferred because it is comparatively less expensive to remove any remaining water and excess binding agent mechanically than thermally. In the present process, a press temperature in the range of 330-350° F. is contemplated.

The intermediate web saturated with binding agent can then be conveyed to a dryer. Water can be removed from the intermediate web by evaporation, thus leaving the binder behind. It is contemplated that any known convection, contact, or radiation dryer would be suitable for this purpose. In the preferred embodiment, conventional steam heated drum dryers are employed due to their availability commercially.

Once dried, a composite web is obtained. The composite web is particularly suitable for further processing into moldable, or pressed consumer or industrial products. The fact that composite web includes a relatively short fiber component imparts the benefit of moldability.

If a low melting point component is used, it can impart the final product with other properties, such as water resistance. If a high melting point component is used, it can improve the structural qualities of the final product. As a result, the inherent qualities of the post-industrial scrap fibers are maximized to impart desirable characteristics to the composite web.

### V. Drying Steps

The non-woven material can then be subjected to a drying step, with the goal of bringing the moisture content down below 18% moisture by weight, and, ideally, to a moisture content of about 3-10% by weight.

### VI. Optional Calendaring Step

The dried, non-woven material can, if desired, be subjected to a calendaring step. The nonwoven substrate, whether it is a Spunlace nonwoven, or TABCW, Coform, airlaid, spunbond, SMS etc., can be unwound, and a dispersion (preferred) or solution of chemical agents, such as HYPOD8510 or 8102.2, additives (e.g. Lutensol A65 N, HPC, cotton linters, silica gel, Unifroth 0154) and water can be froth foamed onto a heated calender roll (also referred to as dryer, heated drum etc.). A heated drum (such as a crepe drum or a cast iron drum) heats the solution and evaporates the water while melting the solids in the solution/dispersion into a thin film-coating on the heated drum. A nip roll can be used to apply pressure to adhere the film to the nonwoven substrate.

A creping/skimming blade can be used to scrape film/nonwoven substrate off of the drum to produce a coated substrate.

The embossing/calendering roll can be used to not only adhere a substrate to the non-woven material, but also to apply calendering patterns and designs to the material.

In some embodiments, the resulting material would be wound up on a winder and later converted to finished goods. However, in other embodiments, the material is subjected to a chemical vapor treatment, as such is described herein.

### VII. Chemical Vapor Treatment

Once the textile/leather scraps have been reduced in size to fibers, the fibers have been appropriately sized, and formed into a non-woven material, the fibers in the non-woven material can be moistened, humidified, and/or lubricated, subjected to an enzymatic treatment, or otherwise chemically treated.

Due to the nature of this technology and its importance in high quality downstream applications, there are many chemical treatments (referred to herein as catalytic vapor treatment, when such treatments are applied in conjunction with steam that operates to open the fibers and aid the treatments in penetrating the fibers) that can be delivered to improve the performance of the non-woven materials.

Collectively, the combination of steam and chemical/enzymatic treatment is referred to herein as “catalyzed vapor treatment.” Although the steam is not technically a catalyst, it opens up the fibers and allows them to receive the chemical/enzymatic treatments more efficiently. The water is added in the form of steam, and is removed as the fibers are later isolated, so is not a reactant per se. For this reason, the process has been termed “catalyzed vapor.”

The treatments can serve one or more purposes, such as softening and relaxing twists in the waste materials, providing stain resistance, providing resistance to microbial contamination, providing color, fiber strengthening aids, oils to aid in anti-static formulation, nanotechnology for finishes on fabrics downstream, antimicrobials to kill microbial contaminants, and surfactants for personal care products.

Chemical treatments and finishes applied in accordance with the method of this invention are typically in the form of fluids (liquids) which enhance or provide a desired functionality to the substrate material, such as softening, hydrophilic, hydrophobic, anti-static, stain-blocking, and stain-resistance properties, to name a few.

Suitable chemical treatments for achieving these functional properties are known to those skilled in the art. Such treatments and finishes can also be used to enhance the structural integrity of the substrate materials or enhance aesthetics, such as by applying dyes and pigments. These chemical treatments and finishes can be applied, if desired, in a registered manner, that is, in a manner by which precise control is maintained over the placement of the materials in accordance with a predetermined pattern or arrangement.

Softening agents are typically added in an amount of between 0.1 and 10 weight percent of the nonwoven web. These chemicals may be any of those commonly known to those skilled in the art as being useful for softening textiles. Softeners may be silicone, anionic, nonionic or cationic though cationic softeners are preferred.

Anionic softeners are generally chemical compounds such as sulfated oils like castor, olive and soybean, sulfated synthetic fatty esters, such as glyceryl trioleate, and sulfated fatty alcohols of high molecular weight.

Nonionic softeners are highly compatible with other finishing agents and are generally compounds such as glycols, glycerin, sorbitol and urea. Compounds of fatty acids like polyglycol esters of high molecular weight saturated fatty acids such as palmitic and stearic acids are other examples.

Cationic softeners are generally long chain amides, imidazolines, and quarternary nitrogen compounds. One suitable cationic softener is a tallow based quarternary ammonium compound sold under the tradename Varisoft®.

Textile softeners are discussed in Textile Laundering Technology (1979), Riggs, C. L., and Sherill, J. C. (p. 71-74), the magazine American Dyestuff Reporter, September 1973 (p. 24-26) and the magazine Textile World, December 1973 (p. 45-46).

Quaternary ammonium compounds can be applied, for example, to suppress odors and control micro-organisms.

The non-woven materials can also be treated with emollients, surfactants and skin care lotions. The term emollient as used herein, refers to the semi-solid or liquid material used to provide a moisturizing, soothing feeling to the skin. Typically, emollients can be soluble or insoluble in water, but are ideally non-volatile under condition of application and use to ensure a durable effect.

Examples of commercially available classes of emollients include, without limitation, hydrocarbon oils and waxes, acetoglyceride esters, silicone oils, ethoxylated glycerides, triglyceride esters, alkyl and alkenyl esters, fatty acids and alcohols and their esters and ethers, lanolin and it's derivatives, waxes derived from natural or synthetic sources, phospholipids and polyhydric alcohol esters. Some common examples are Aloe Vera, petrolatum, mineral oil, essential oils, hydroxy fatty acids, mono-, di- and tri-glycerides, esters and amides of fatty acids and the like. Particularly suitable emollients are mineral oil, petrolatum, vegetable oil, paraffin oil, and silicone oils.

Optionally, the emollient can contain a functional amount of surfactant. As used herein, surfactant refers to liquid, semi-solid or solid products used to provide compatibility between the finish and coating component in the formulation. Surfactants may also provide emulsification of the emollient and modify the hydrophobic properties of the fibrous substrate by allowing rapid transport of aqueous liquids. Classes of surfactants useful for this invention are listed below. The mixture of emollient and surfactant is typically referred to as the finish and generally will contain from about 5 to about 90% of the emollient with the remainder being one or more surfactants.

The oily mobile material of this invention may also primarily comprise a surfactant. Non-limiting examples of types of surfactants suitable for use in the present invention are sulphonates of alkanes and alkenes, salts of long chain fatty acids, ethoxylates of amines, alcohols, polyols and acids, alkoxylates, fatty acid esters, phosphate and sulfonate esters, sulphosuccinates and sulphosuccinamates, aryl sulphonates, castor oil ethoxylates, glycosides, protein derivatives, various block co-polymers, and mixtures thereof.

The treatment compositions in the catalyzed vapor treatment can include one or more of an enzyme, such as a cellulase, protease, lipase, pectinase, or amylase enzyme, a surfactant, which can be a cationic, anionic, zwitterionic, or nonionic surfactant, or a silicone treatment. The selection of the treatment depends on the type of textile materials to be treated.

In the case of rayon fabrics, the catalyzed vapor can include a softener, which can be, for example, a blend of a non-ionic softeners such as alkyl polyethanoxyether or a polyoxyethylene alkyl ether. However, the application of water is not as critical here.

Products which have proven to be successful as part of the catalyzed vapor technology for these fabrics have been Perrustol CCF, Perrustol CCA or Softycon's RWT.

For example, in the case of cotton, chemical treatments often include starches, waxes, anti-crease finishes, hydrophobic finishes, and the like. In the case of synthetics, the finishes often include polycarboxylic acids (PCA), acrylic sizing agents, oiling waxes or silicone based lubricants, and combinations thereof.

Representative strengthening agents include Para-KRC from the Kunal Group, which can increase tear and tensile strength for silk.

Multiple surfactants can be used for cotton, and a product similar to Avco-Soft PE is excellent when catalyzed for PVA in this area of the process.

A product using a poly (vinylamine-vinylformamide) copolymer with a carrier can be used to improve the strength of fibers that have been weakened, for example, as a result of mechanical stress in the case of a natural hair collection such as cashmere.

These strengthening treatments can be used to increase the tensile strength of the treated fibers, relative to untreated fibers.

In one embodiment, a further treatment includes the application of an antimicrobial agent, which is particularly useful if the downstream product is going to be used in the healthcare, medical, personal care or baby products industries.

If in a downstream application it is necessary for a particular non-woven to have a hydrophobic property, or to have a more hydrophilic nature, a suitable treatment can be applied.

Further treatments include, but are not limited to, fragrances, colorants, fillers, essential oils, vitamins, antibiotics, and the like.

Lubrication creates drape, softness and strength. For example, where the fibers are leather fibers, it is relevant to note that leather in its natural state is a non-woven material where the fibrils of the fiber have grown together. After fiberization, the natural leather has been deconstructed. In the rejuvenation of this product, it is advantageous to reconstruct the semblance of nature by returning the fibers to a natural non-woven material.

Where one or more components of a chemical treatment or finish are solids, the components can be heated until all have melted, stirred until the mixture is homogenous, then applied to the non-woven material.

In use, the textile materials to be treated are passed through the hopper or feedbox onto a first conveyor, and then into a treatment station, where the fibers in the textile materials are opened up by exposure to steam, and then treated with one or more treatment compositions. Within any given treatment station, one or more treatments can be applied. The treatment compositions can be in the form of a vapor, droplets, a stream of liquid, a dispersion, and the like, though vapor can be preferred.

The treatment(s) can be applied to the textiles using one or more spray nozzles located above the textile materials to be treated, which in some embodiments move along a conveyor belt and pass under the spray nozzles. As and after a treatment is applied, whether as a vapor, solution, dispersion, spray, and the like, a vacuum can be pulled below the conveyor, or above the textile to be treated, respectively, pulling the treatment through the textile to be treated. Excess treatment chemicals can be collected and recycled, if desired.

The treatment composition can include, for example, a surfactant, a silicone treatment, depending on the type of textile materials to be treated; an organic agent which strengthens yarn element of the textile materials for further processing, e.g. a surfactant in the case of deconstructing a cotton fabric, an anti-microbial application or a biocide.

A catalyzed silicone vapor could be one of the many selections used for cotton in this instance such as Softycon's SHP-C, Sofytcon's TRN or Rexamine CP 9194 AL. Other choices include cellulase enzymes, surfactants or other silicone softening treatments.

Before the chemicals/enzymes are applied, the fibers can be opened up by applying high temperature steam. Ideally, the steam is hot enough that the residual heat evaporates the bulk of the water, and the fibers do not absorb sufficient water to raise their moisture content above around 25% by weight, more typically around 15% by weight. In some embodiments, the steam is superheated steam, and in other embodiments, the steam is not superheated steam.

The high temperature steam is ideally applied for a period of time sufficient to destroy microbes that might be present.

In another embodiment and as appropriate to the product, an alcohol, such as isopropyl alcohol, is used an alternative to steam.

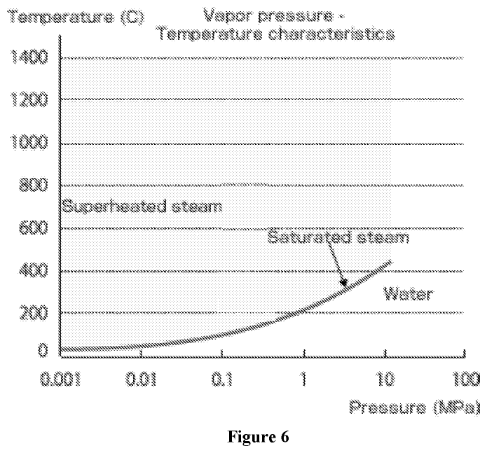

As used herein, high temperature steam is steam that is hotter than the boiling point of water. Those of skill in the art can readily appreciate that there is a difference between saturated steam, slightly superheated steam and superheated steam, any of which can be used to treat the fibers and/or non-woven materials, and, optionally, to disinfect them. In one embodiment, the high temperature steam can be saturated steam, superheated steam, and anything in between. FIG. 6 is a chart showing the correlation between the temperatures and pressures of the different types of steam, and those of skill in the art can select appropriate temperatures and pressures at which to treat the fibers and/or non-woven materials.

Superheated steam is “dry.” Dry steam typically must reach much higher temperatures and the materials exposed for a longer time period to have the same effectiveness; or equal FO kill value. However, slightly superheated steam can be used for antimicrobial disinfection (see, for example, Song, L.; Wu, J.; Xi, C. (2012). “Biofilms on environmental surfaces: Evaluation of the disinfection efficacy of a novel steam vapor system”. American Journal of Infection Control 40 (10): 926-930).

The fibers and/or non-woven materials can be treated, and, optionally, disinfected, with saturated steam, superheated steam or slightly superheated steam, as those terms are understood by those of skill in the art. In one embodiment, the fibers and/or non-woven materials are passed through a “steam disinfecting module.”

In one embodiment of a substantially closed system, the steam, whether saturated steam, slightly superheated steam, or superheated steam, is recirculated. Any steam that condenses during the process can optionally be discharged from the device.

Ideally, the contact time is such that the moisture content of the fibers and/or non-woven materials does not exceed about 15% by weight. If this moisture content is not exceeded, the materials may not need to be dried. If they are to be dried, the conditions of drying are selected subject to the product. Those skilled in the art can readily determine a suitable optimization on the basis of their normal expert knowledge. In most of the cases, the temperature will range between 150 and 500° C. The drying, if performed, can be carried out in a drying chamber. The steam passing through the fibers and/or non-woven materials, and, optionally, the steam coming from the drying chamber, can again be compressed and heated to a desired temperature and pressure, and returned to the steam disinfecting module.

The injected steam ideally is food grade, and therefore in essence free from mineral oil, moisture droplets, microorganisms, and dirt.

As the steam is applied to the fibers and/or non-woven materials, such as through a nozzle, atomization and disinfection, and, in some embodiments, sterilization, takes place simultaneously, and in a substantially closed system the excess steam can be reused.

The steam, in one embodiment, is provided by a steam generator, which boils, for example, distilled or deionized water. In one aspect of this embodiment, the water is degassed prior to use, so that the steam contains few impurities and almost no non-condensing impurities.

The steam generator may be at any temperature above the final temperature, e.g., 150° C., as the thermal treatment of the droplets derives mainly from the latent heat of vaporization of the droplets, and very little from the absolute temperature of the steam. Preferably, the steam is saturated, which will define its temperature in a given atmosphere.

The steam can be derived from a boiler. Temperature control can require a high temperature boiler with a control valve near the point where the steam is applied to the fibers and/or non-woven materials. In other words, in order to ensure adequate flow of steam, an excess capacity should be available from the boiler. Control is effected near the point of application, to avoid time response delays or oscillation. The water in the boiler is preferably degassed to eliminate non-condensable components. The boiler can optionally include a superheater at its outlet, to heat the steam over a condensation equilibrium level.

The steam can injected into a steam disinfection module through one or more steam injection ports/nozzles. Where two or more ports/nozzles are used, they can be spaced within the chamber, so that the region distant from the fluid injection port maintains a relatively constant water vapor pressure. The walls of the reactor vessel should be maintained at least at or slightly above the final operating temperature, to avoid condensation of steam on the wall s of the module. This may be accomplished by any suitable heating system.

The steam sterilization module can use a controlled dry steam cycle for a predetermined temperature at a predetermined pressure based on the type of fiber/non-woven material, the batch size, and other relevant factors. For example, the controlled dry steam cycle may be for 5-300 minutes at between 100 to 300° F. at 0-15 psi, more specifically, for 15-25 minutes at around 150° F. at around 5 psi.

Polymers

The polymers useful in preparing the non-woven materials described herein, whether they are applied at this stage, or in the calendaring stage, or both, include thermoplastic polymers and thermoset resins, and the polymers can be present in the form of a latex dispersion. Natural materials, such as natural latexes and rubber, can also be used, as can synthetic polymers.

Representative thermoplastic polymers include polyolefins, such as polyethylene, polypropylene, and copolymers thereof, polyvinyl alcohol, polyethylene/vinyl alcohol, polyvinyl acetate, polyvinyl alcohol, poly(meth)acrylate, poly(meth)acrylic acid, polymethyl(meth)acrylate, polyvinyl acetate, polyvinyl chloride, poly ethylene/vinyl acetate, poly ethylene/vinyl chloride, neoprene, polystyrene, polystyrene-co-acrylate, polystyrene/butadiene, polystyrene/acrylonitrile, polybutadiene, poly butadiene/acrylonitrile, polyacrylonitrile/butadiene/styrene (AB S), poly ethylene/acrylic acid, polyethylene/urethanes, polycarbonates, polyphenylene oxides, polypropylene, polyamides such as nylon, polylactide/polylactic acid (PLA), polybenzimidazole, polycarbonate, polyether sulfone, polyether ketone, polyetherimide, polyphenylene oxide, polyphenylene sulfide, and polytetrafluoroethylene (Teflon).

Acrylic polymers, including poly(methyl methacrylate) (also known as PMMA, Lucite, Perspex and Plexiglas) is found in aquariums, motorcycle helmet visors, aircraft windows, viewing ports of submersibles, lenses of exterior lights of automobiles, and signs, including lettering and logos.

Acrylonitrile butadiene styrene (ABS) is a light-weight material that exhibits high impact resistance and mechanical toughness, and is found in many consumer products, such as toys, appliances, and telephones.

Nylon and other polyamides are usually used as inexpensive substitutes for hemp, cotton and silk, in products such as parachutes, ship cords and sails, flak vests and women's stockings. Nylon fibers also found in fabrics, rope, carpets and musical strings, and in bulk form, nylon is used for mechanical parts including machine screws, gear wheels and power tool casings. Nylon particles can be used to form heat-resistant composite materials.

Polylactic acid (polylactide, PLA) is a biodegradable thermoplastic aliphatic polyester derived from renewable resources, such as corn starch (in the United States), tapioca roots, chips or starch (mostly in Asia), or sugarcane. It is one of the materials used for 3D printing with fused deposition modeling (FDM) techniques.

Polybenzimidazole (PBI) fibers have a very high melting point. It has exceptional thermal and chemical stability, does not readily ignite, and is highly stable. Polybenzimidazole is found in high-performance protective apparel such as firefighter's gear, astronaut space suits, high temperature protective gloves, welders' apparel and aircraft wall fabrics, as well as membranes in fuel cells.

Polycarbonate (PC) thermoplastics are known under trademarks such as Lexan, Makrolon, Makroclear, and arcoPlus. They are found in many items, such as electronic components, construction materials, data storage devices, automotive and aircraft parts, check sockets in prosthetics, and security glazing.

Polyetherimide (PEI) has high heat distortion temperature, tensile strength and modulus, and is generally used in high performance electrical and electronic parts, microwave appliances, and under-the-hood automotive parts.

Polyethylene (polyethene, polythene, PE) is a family of similar materials categorized according to their density and molecular structure. Ultra-high molecular weight polyethylene (UHMWPE) is tough and resistant to chemicals, and is commonly found in moving machine parts, bearings, gears, artificial joints and some bulletproof vests. High-density polyethylene (HDPE), is commonly found in milk jugs, liquid laundry detergent bottles, outdoor furniture, margarine tubs, portable gasoline cans, water drainage pipes, and grocery bags. Medium-density polyethylene (MDPE) is commonly found in packaging film, sacks and gas pipes and fittings. Low-density polyethylene (LDPE) is flexible, so is commonly found in squeeze bottles, milk jug caps, retail store bags and linear low-density polyethylene (LLDPE) is used as stretch wrap in transporting and handling boxes of durable goods, and in common household food coverings. XLPE or “PEX” (cross-linked polyethylene) is a semi-rigid, flexible material used in cold or hot water building heating and cooling applications (hydronic heating and cooling) due to its exceptional resistance to breakdown from wide temperature variations.

Polyphenylene sulfide (PPS) has outstanding chemical resistance, good electrical properties, excellent flame retardance, low coefficient of friction and high transparency to microwave radiation. PPS is principally used in coating applications, and in injection/compression molding applications, and found in cookware, bearings, and pump parts for service in various corrosive environments.

Polypropylene (PP) is found in diverse products, such as reusable plastic food containers, microwave- and dishwasher-safe plastic containers, diaper lining, sanitary pad lining and casing, ropes, carpets, plastic moldings, piping systems, car batteries, insulation for electrical cables and filters for gases and liquids. Polypropylene sheets are used for stationery folders and packaging and clear storage bins.

Polyvinyl chloride (PVC) is a tough, lightweight material that is resistant to acids and bases. It is commonly found in materials used by the construction industry, such as vinyl siding, drainpipes, gutters and roofing sheets. It is also converted to flexible forms by adding plasticizers, and found in items such as hoses, tubing, electrical insulation, coats, jackets and upholstery. Flexible PVC is also found in inflatable products, such as water beds and pool toys.

Teflon has excellent properties for low friction and self-lubrication, and is commonly found in the airplane industry in parts such as bearings/pins, clutches, bushings, brace bearing brackets, duct supports in the engine, slats, gear retraction actuators, slat bearings, flaps levers, flaps torque tube bearings, control actuator elevators, and rudder ring/trunnion bearings.

Optional Components

The optional components which can be added are not limited to narrow categories. Furthermore, one or more of the following can be added as desired. When added, multiple types may be adhered to the same fibers in the composite materials. Also, the specific examples listed in the categories identified below are by no means exhaustive, nor are the categories intended to be limiting for the purpose of the present invention.

The composite materials described herein can optionally include one or more pigments or colorants as desired. Pigments or colorants can broadly be defined as being capable of re-emitting light of certain wavelengths while absorbing light of other wavelengths and which are used to impart color. Charcoal can be added, both as a colorant and as an adsorbent.

Fire retardant materials can also be added. Fire retardants are those which reduce the flammability of the fibers in the composite web. Preferably these materials are active fire retardants in that they chemically inhibit oxidation or they emit water or other fire suppressing substances when burned.

Although not limited to specific materials, examples of suitable materials include pigments and whiteners, such as inorganic pigments including titanium dioxide, ferrous oxide, PbO, Al₂O₃ and CaCO₃ and organic pigments or colorants, ultraviolet, infrared or other wave length blocking or inhibiting particulates, such as carbon blacks as an ultraviolet inhibitor and zirconium carbide as an infrared inhibitor; fire retardant materials, such as alumina trihydrate, antimony oxide, chlorinated and brominated compounds, pentabromochlorocyclohexane, 1, 2-Bis 2, 4, 6-tribromophenoxy ethane, decabromodiphenyl oxide, molybdenum oxide and ammonium fluoroborate, and the like.

The non-woven materials can optionally also include one or more pesticides, insecticides, fertilizers, antimicrobials, such as broad spectrum antimicrobials (e.g. hypochlorites, perborates, quaternary ammonium compounds such as benzalkonium chloride, bisulfites, peroxides, etc.), antivirals, antimycotics, antibacterials, antirickettsials, antibiotics, biocides, biostats, etc., and mixtures thereof.

VIII. Disinfection and/or Sterilization

The non-woven materials, after having been subjected to a chemical vapor treatment, may still have residual microbial contaminants. This residual contamination can be addressed by exposing the products to one or more of UV light, ethylene oxide, and/or methyl bromide, at a sufficient concentration, and for a sufficient time, to destroy microbes that might be present. Thus, the non-woven materials can be disinfected, and, ideally, sterilized at this stage. This can be accomplished, for example, using UV light, preferably UV-C light.

The incoming fibers or the non-woven textile materials formed using the processes described herein can be exposed to UV-C radiation, which can accomplish one or more of the following:

(1) to pre-sterilize the fiber or non-woven materials in order to rid them of bacteria associated with the system or human contamination;

(2) to modify the surface of the fiber or non-woven materials in order to increase their wettability and absorbability for the purpose of downstream production or products; and

(3) to modify the surface of the fiber or non-woven materials in order to eliminate the pilling issues that create neps in the rejuvenation of natural fibers.

The UV radiation, such as UV-C radiation, penetrates the entirety of the fibers/non-woven textile materials in order to optimize pre-sterilization. Pre-sterilization of the textile materials is important to ensuring quality materials for a number of downstream applications created from rejuvenated textile materials. Applications such as pharmaceutical, medical, baby, cosmetic, or food grade products require levels of microbial testing of the textile materials prior to downstream non-woven or textile processing. This is the initial area where the removal of those microbes will occur.

Utilizing UV-C is a powerful step when used in conjunction with this methodological approach. Exposing the textile materials to the UV-C radiation can rid the materials of bacteria associated with the earlier stages of processing and the human contamination that has occurred upstream. The UV-C radiation is typically applied at a wavelength between 100 nm and 280 nm for this type of disinfection. This is yet another critical step in assuring a quality rejuvenated fiber in downstream applications. The exposure of textile materials to UV-C radiation in step (c) also has an effect on some of the physical and mechanical properties of the fibers and/or non-woven textile materials.

While the UV-C radiation is sterilizing the fibers and/or non-woven textile materials, it is also modifying the surface of the fibers/materials to create greater hydrophobicity. Due to the modification of the surface of the fibers/materials, nep counts can be reduced. Pilling can also be significantly reduced due to this surface modification.

The UV irradiation of the textile materials also affects the color strength of the textile materials. Previous studies show that UV-C irradiation adds value to coloration and also increases the dye uptake ability of cotton fabrics through oxidation of surface fibers of cellulose. UV or gamma are ionizing radiations that interact with the material by colliding with the electrons in the shells of atoms. They slowly lose their energy in material and are able to travel significant distances before stopping. The free radicals formed are extremely reactive, and they will combine with the material in their vicinity. Upon irradiation, the cross linking changes the crystal structure of the cellulose, which can add value in the coloration process and causes photo modification of surface fibers. The irradiated modified fabrics allow an increase in the wettability of hydrophobic fibers, which improves the uptake of organic process chemicals used to eradicate surface chemicals in the next process of rejuvenation. It also has a positive effect in improving the uptake of the dyestuffs and will increase the depth of shade in downstream printing and dyeing.

The below table shows the results of tests conducted according to ASTM C5866-12.

| | | | Post UVC |
|---|---|---|---|
| | | Post-UV-C | Treatment |
| | Pre UV-C | Treatment/ | in Yarn |
| Cotton | Treatment | Fiber | Application |
| Test 1 | 870 neps/gram | 430 neps/gram | 60 neps/gram |
| Test 2 | 1100 neps/gram | 480 neps/gram | 72 neps/gram |
| Test 3 | 1286 neps/gram | 760 neps/gram | 73 neps/gram |

Tests above were performed on a High Speed Fiber Testing Unit, AFIS to determine nep count in the rejuvenated cotton fibers which uses a sliver of cotton fed into the automated unit to determine how many neps per gram of fiber that was detected.

In one embodiment, UV light is applied in a tunnel. The tunnel can optionally include reflective sides, top and bottom. In another embodiment, before being baled, or before being converted to non-woven materials, the fibers themselves can pass through an additional tunnel with similar application of UV light. Thus, the fibers and/or the non-woven materials can be treated to remove microbial contaminants.

In one embodiment, a UV chamber includes a mirrored top, bottom and sides to enhance the bulbs, although the chamber can be effective without using mirrors. The chamber can have a width in the range of about ½ meter to about 7 meters, though wider and narrower chambers are acceptable. The lengths are typically in the range of about 1.2 meters to about 9 meters, though longer or shorter lengths can be used. The chambers can be vertical or horizontal, though horizontal can be preferred due to the ease of service.

The time in the tunnel is determined by what bacteria are to be killed, and how wide and how narrow the tunnel is. Typical residence times are from a few seconds to 30 minutes.

Typically, the fabric is between about 3 and about 14 inches from the UV bulbs. The number of bulbs will depend, of course, on the width and length. Ideally, the wavelength of light used is less than about 300 nm.

Alternatively, or additionally, ultraviolet disinfection, ozone disinfection, microwave sterilization, electron beam sterilization, magnetic sterilization, resistance heat sterilization, pasteurization, UHT (UHT), radiation high pressure sterilization, ultrasonic sterilization, hydrogen peroxide sterilization, exposure to ethylene oxide, or exposure to methyl bromide can be used to disinfect and/or sterilize the fibers and/or non-woven materials.

### IX. Optional Waste Classification

The raw materials can be amassed and identified using a raw material data system (RDS) and specially trained personnel. Regional team members begin by gathering information and documentation from the various sources materials where materials are to be collected.

This information can be used to initially categorize and identify the composition of the types of material these sources produce. This information also provides the amount of estimated production and percentage of waste that will be generated over a forecasted period of time.

Material can be gathered at the location site, where it is then placed in appropriate vessels such as bags, bales, boxes, and the like for storage in enclosed trailers, shipping containers or other secure means of transport.

Once it departs the origin manufacturing site, it can be transported to a main collection facility. At each collection facility, it can undergo identification and quality checking before undergoing further processing.

Representative identification and verification steps include documentation verification, testing methods such as solvent extraction, microscope, stain, flame, FTIR, and other standard testing methodologies.

Once confirmed and classified, the material can be repackaged according to the composition, labeled and temporarily stored.

Each bale or material container can be identified by unique codes specific to their contents. The approximate composition of the material can be determined through testing methods such as solvent extraction or FTIR. The results can be compared to the original documentation for confirmation it was correctly reported.

Where the material is not homogeneous, but rather, includes multiple waste components, it can be catalogued, for example, by listing the percentages of the various components. In one aspect, the percentages are listed in ascending order, and in another aspect the percentages are listed in descending order. For example, carpet waste may contain 50% wool, 40% sisal, 5% olefin, and 5% latex. In this example, the largest percentage is wool, and the secondary highest is sisal.

In some embodiments, it may be desirable to use the materials as is. In other embodiments, it would be desired to use materials with more or less of a given component. However, in other embodiments, it can be advantageous to blend bales of waste material to arrive at a desired content. For example, where a recipe for a composite material involves using a raw material with an olefin content closer to 30%, and a relatively lower amount of sisal from the above described bale, one can combine the first bale with a second bale where the olefin content is higher and the sisal content is lower, including bales which contain no sisal. By combining bales, one can arrive at a relatively uniform mixture with a ratio of components at or near what is desired for a given “recipe.”

Thus, by identifying the content of waste materials to be recycled, one can strategically create “blending recipes” to create the core materials needed for producing a variety of composite materials, including structurally-sound moldable substrates. The combination of blend variations and manner in which they are increased or decreased relies heavily on these qualifying tests by the collection facility. There are numerous combinations of blends of materials that can be collected, and the formulation of blends can be simple or complex, depending on the requirements of the final blend needed.

Some of the components of these materials are less than 5% of the total, and can be considered to be “enhancements” to the resulting blend, and when they are in particulate form, they can be considered to be “enhancement particles.” At relatively low concentrations, it may or may not be necessary to include these components in the list of components for each bale, and may or may not be relied upon to achieve the desired properties of the final composite material. That said, where such materials could interfere with the downstream processing, or if a restriction is stated by the end customer, or a city, state, or country governing body, it may be necessary and appropriate to list the materials. In some embodiments, it is appropriate to list even minor amounts of certain components on a material safety data sheet (MSDS). However, at relatively low concentrations, the components typically have little or no impact on the properties of the final product, as the relative percentage of the material is often decreased in further process steps as other components are added.

At a processing location, quality control verification steps can be used to confirm that the list of components for each bale of material is correct. The materials can be registered into inventory, and stored until such time as they are used for further processing. In some instances, moisture levels, absorption testing, and other specified types of testing are performed to ensure the quality of the material will meet the standards required for downstream processing. These quality control practices not only verify quality, but also aid the traceability of material to its origin, which can be of particular importance in terms of various qualifying credits, where applicable.

Each group of material can have a source code to identify its source or origin as well as its composition.

As an example, once a source of leather, such as post-industrial or post-consumer waste leather materials is obtained, the process can first involves obtaining data on the type of polymer coating applied to the leather, so as to facilitate its removal. Data can also be obtained on the types of treatments or finishes that the incoming waste leather may have received during production, as well as data on the color and shade of leather.

One way to determine the type of polymer coating on the leather involves FTIR (fourier transform infrared) spectroscopy. The FTIR can be performed by dissolving the polymer in a solvent, then removing the solvent to yield a polymer. If the polymer is too opaque, it can be crushed into a powder, mixed with potassium bromide, and formed into a thin disk for use in generating an FTIR scan. Another way to perform FTIR is to use reflective FTIR, where the IR passes only a few microns into a surface to be tested. Still another way is to use an abrasive that does not absorb light in the desired portion of the IR spectrum to scratch the polymer surface, then to perform an FTIR screen on the abrasive surface.

The spectrum can be stored, if desired, in a computer database. Ideally, the spectrum is screened against a library of other spectra, and the type of polymer can be identified by computer matching. While the exact member of a class of polymers may not be identified, typically each polymer type will provide an FTIR spectra with certain key peaks, making it possible to identify the type of polymer coating on the leather.

In this manner, one can obtain data for each bale of incoming waste leather, related to the type of coating on the leather, and this information can be stored in a database.

Similar assessments can be performed on other types of waste materials.

### X. Winding Up and Further Processing of the Non-Woven Materials

The non-woven materials, after having been treated to remove microbial contaminants, can be wound up and stored for later use, or converted to final products.

Due to the purification technology in this process, an outside sourced spunbond non-woven material can be un-wound prior to the spunlace process and added to the spunlace web for unique web and strength formation of the product without sacrificing purity of the product. As long as the outside sourced raw material is added prior to purification the body of the product will be absent of microbial contamination. For example, a spunbond nonwoven fabric can be spread over a conveyor at the bottom part of a roller spreader, and desired chemicals can be applied to the nonwoven fabric.

The overlaid product thus obtained can be sandwiched from the top side with an air-through nonwoven fabric as a water-permeable nonwoven fabric, and thereafter heat-fused with a laminating machine, to give a water-absorbent sheet structure. The resulting water-absorbent sheet structure can be cut into a given size with a water-permeable nonwoven fabric

The present invention will be better understood with reference to the following non-limiting examples.

### Example 1: Material Processing Flow

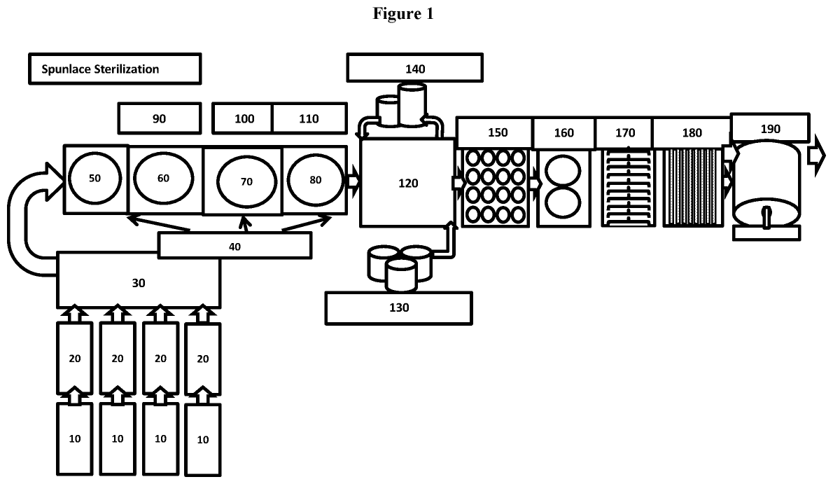

An example of one embodiment of how materials can flow throughout the process described herein is shown in FIG. 1.

To start the process, baled fiber-based materials can be fed, for example, via a robotic loader, from a series of bales (10) into a blend line (20), and then conveyed to a blend line storage box (30), where the fibers can be intimately blended.

The blended fibers are conveyed, using a web conveying system (40) to an airlaid card (50), and then to a first non-woven card (60), a second non-woven card (70), and a third non-woven card (80), to align the fibers. The web conveying system includes a first web conveyor (90) to convey the material from the airlay card to the first nonwoven card, a second web conveyor (100) to convey the material from the first nonwoven card to the second nonwoven card, and a third web conveyor (110) to convey the material from the second nonwoven card to the third nonwoven card. From there, the material is conveyed to a spunlace system (120) with a dewatering belt. In those embodiments where it is desired to apply an organic chemical treatment to the spunlace non-woven material, a chemical dosing system (130) is present. To reduce microbial contamination, water used in the spunlace process is filtered through a water filtration system (140), which ideally includes filters with a pore size less than 0.5 microns, and, more preferably, less than about 0.2 microns, to minimize bacterial contamination.

The spunlace nonwoven material is then dried in one or more drying cans (150), and then calendared (160). From there, the non-woven material is then treated with superheated steam so as to destroy any bacterial contaminants. The steam is applied in a module (170) which includes one or a plurality of nozzles through which the superheated steam can be applied. Because the steam is superheated, it quickly evaporates from the treated spunlace nonwoven material. From there, the material is transported to a module which applies UV light in a sufficient amount and for a sufficient duration of time to effectively destroy any residual contamination. From there, the material can be rolled up on a winder (190) and stored until it is ready to be converted to final products.

In one embodiment, not shown, the material is later cut into individual sheets, provided with a baby-safe cleaning solution, and used as baby wipes. In one aspect of this embodiment, the baby wipes are made from a material that includes post-industrial and/or post-consumer cotton fibers.

### Example 2: Exposure of Non-Woven Webs to UV Light

Non-woven webs with a density of 27 g/ft² were evaluated in a UV light system, in an effort to identify conditions suitable for decontaminating non-woven webs. The webs were contaminated with a variety of microbes, including E. coli, Staph. aureus, Pseudomonas aeruginosa, Candida albicans, Salmonella enteritidis, Bacillus subtilis, and Aspergillus niger spores.

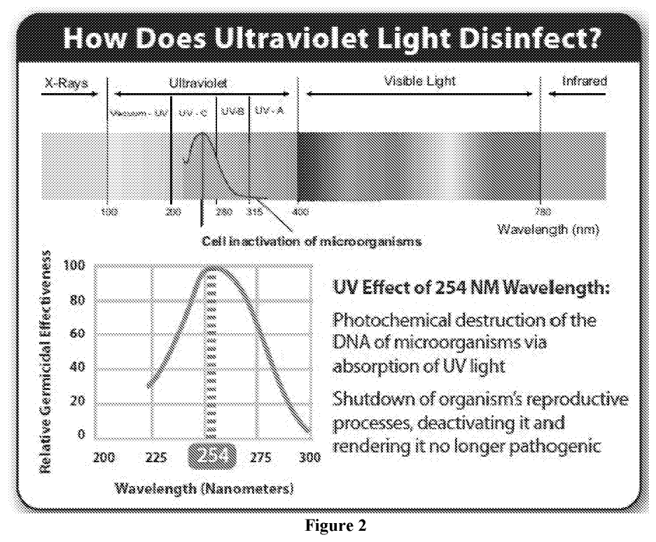

As shown in FIG. 2, UV light, particularly light in the UV-C range, is known to have antimicrobial properties. However, it is also known to damage textiles and fibers when the intensity of the light is too high.

In an effort to obtain an antimicrobial effect without damaging the non-woven materials that were being evaluated, the UV lights were set a certain distance, namely, one foot, from the materials. However, satisfactory results are generally obtained when the distance is between about 6 inches to about 2 feet from the non-woven materials, or fibers, to be treated.

A medium intensity UV-C lamp (12 lamp 540W output) system was used, and the exposure time was 3.7 seconds, although a more typical range of exposure times is around 4 to around 7 seconds. A mirrored backing was used to bounce back the light onto the substrate, in order to enhance the effect, though such is not necessary.

The results are tabulated below.

Microbial Growth Control Summary from test model:

| | Seconds for | Seconds for |
|---|---|---|
| Microbes | 90% Kill | 99% Kill |
| Escherichia Coli | 0.45 | 0.89 |
| Staphylococcus aureus | 0.73 | 1.47 |
| Pseudomonas aeruginosa | 0.41 | 0.82 |
| Candida albicans | 9.26 | 18.52 |
| Salmonella enteritidis | 0.24 | 0.47 |
| Bacillus subtilis | 1.48 | 2.96 |
| Aspergillus niger spores | 36.07 | 72.13 |

In summary, the UV treatment is extremely effective at disinfecting, and in sterilizing, non-woven materials.

### Example 3: Representative UV Sterilization Chamber

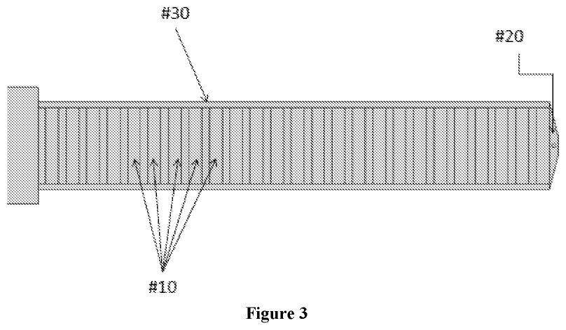

As illustrated in the accompanying drawings, the present invention also provides an apparatus for implementing the aforementioned method utilizing UV-C for treating fiber and/or non-woven materials. A representative UV-C treatment module (also referred to as a UV sterilization chamber, or sterilization chamber) is shown in FIGS. 3-5.

FIG. 3 shows a top view of a UV-C treatment module. A conveyor belt (not shown) moves fibers and/or non-woven materials through the module. UV lights (10) are present throughout the module, and a suction hood (20) is located at the end of the module where the fiber and/or non-woven materials exit the module. The UV lights are housed in an enclosure (30).

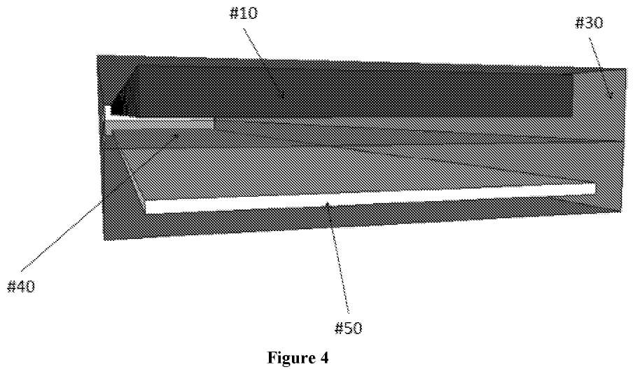

FIG. 4 shows a front view of a UV-C treatment module. A conveyor belt (50) passes under UV lights (10) located in an enclosure (30). In this embodiment, a transparent protective layer (40) is present over the lights to prevent contamination of the fibers and/or non-woven materials in the event one or more of the UV bulbs breaks.

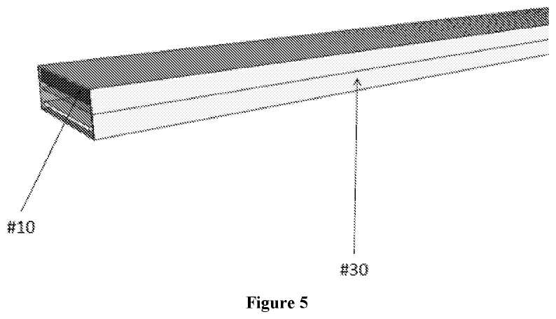

FIG. 5 shows a side view of a UV-C treatment module. In this figure, the conveyor belt is not shown. The enclosure (30) houses a series of UV lights (10) at the top, and the fibers and/or non-woven materials pass under the UV lights (10).

The sterilization chamber receives fibers and/or textile materials, and is disposed with a UV-C radiation source therewithin. The UV-C radiation source irradiates the sterilization chamber with UV-C radiation, which disinfects, and, optionally, sterilizes the fibers and/or textile materials. In some embodiments, it can also modify the surface of the fibers and/or non-woven textile materials, for example, to increase wettability, absorbability and reduce pilling. The UV-C radiation source delivers UV-C radiation with a wavelength between 100 nanometers and 280 nanometers, and can be a pulsating or constant UV-C light source.

The sterilization chamber can, in one embodiment, be in form of a rotating cylinder having an input end and an output end, wherein the fibers and/or textile materials traverse from the input end to the output end as they are irradiated by the UV-C radiation source. The rotating cylinder is approximately 1-2 meters in diameter and 3-5 meters long, and is mounted on a stand with an effective slope of −0.12 to −0.16.

The cylinder can have an interior surface which is highly reflective, and which can be disposed with a plurality of rows of opening slats which are angled with a travel axis along which the fibers and/or non-woven materials travel in the rotating cylinder. The rows can be offset from each other.

The opening slats can also be made of a highly reflective material, and can assist in the opening or “unfolding” of the textile materials while the materials are being tumbled in the rotating cylinder.

Thus, the present invention is well adapted to carry out the objects and attain the ends and advantages mentioned above as well as those inherent therein. While presently preferred embodiments have been described for purposes of this disclosure, numerous changes and modifications will be apparent to those skilled in the art. Such changes and modifications are encompassed within the spirit of this invention as defined by the appended claims.

1. A process for disinfecting, and, optionally, sterilizing, non-woven materials, comprising:
a) subjecting non-woven materials to high temperature steam, in a manner in which the water content of the non-woven materials does not exceed around 25% by weight, where the high temperature steam can be saturated steam, slightly superheated steam, or superheated steam, and
b) subjecting the steam-treated non-woven materials to a disinfecting treatment, wherein the disinfecting treatment is selected from the group consisting of UV light, ethylene oxide, methyl bromide, and supercritical or subcritical carbon dioxide, under conditions sufficient to remove any microbial contaminants that may be present.

2. A process for disinfecting, and, optionally, sterilizing, non-woven materials, comprising:
a) subjecting non-woven materials to high temperature steam, in a manner in which the water content of the non-woven materials does not exceed around 25% by weight, where the high temperature steam can be saturated steam, slightly superheated steam, or superheated steam, and
b) subjecting the steam-treated non-woven materials to a disinfecting treatment, wherein the disinfecting treatment comprises treating the materials with a sufficient amount of ozone to remove any microbial contaminants that may be present.

3. The process of claim 1 or 2, wherein the water content of the non-woven materials does not exceed around 15% by weight.

4. The process of claim 1 or 2, wherein the disinfecting treatment is UV light.

5. The process of claim 4, wherein the UV light is UV-C radiation at a wavelength between 100 nanometers and 280 nanometers.

6. The process of claim 1 or 2, wherein the disinfecting treatment is an ozone treatment.

7. The process of claim 1 or 2, wherein, before or after being subjected to high-temperature steam, one or more chemicals are applied to the non-woven materials,
wherein the chemicals are selected from the group consisting of pesticides, insecticides, fertilizers, antimicrobials, antivirals, antimycotics, antibacterials, antirickettsials, antibiotics, biocides, biostats, and mixtures thereof.

8. The process of claim 7, wherein the antimicrobials are quaternary ammonium salts.

9. The process of claim 1 or 2, wherein the non-woven materials are prepared from virgin fibers, fibers obtained by deconstructing post-consumer and/or post-industrial waste, or combinations thereof.

10. The process of claim 9, wherein the post-consumer and/or post-industrial waste comprises used textiles and/or leather materials.

11. The process of claim 9, wherein the fibers are cellulosic fibers.

12. The process of claim 11, wherein the cellulosic fibers comprise wood fibers.

13. The process of claim 9, wherein combinations of fibers are used, and wherein the combinations of fibers are subjected to an intimate mixing step to provide homogeneity to the fibers, such that the composition of the blend is fairly uniform, wherein fairly uniform means that the composition varies by no more than 20% in any portion of the blended fibers.

14. The process of claim 1 or 2, wherein the non-woven materials are prepared by hydroentanglement or needlepunching prior to being subjected to the high-temperature steam and disinfecting treatment.

15. The process of claim 1 or 2, wherein the non-woven materials are prepared by bonding fibers using thermal, chemical, or adhesive bonding processes.

16. The process of claim 15, wherein the non-woven materials are calendered after the hydroentanglement or needlepunching step, and prior to being subjected to the high-temperature steam and disinfecting treatment.

17. The process of claim 1 or 2, wherein the high-temperature steam is saturated steam or slightly superheated steam.

18. A process for disinfecting, and, optionally, sterilizing, fibers, comprising:
a) subjecting fibers to high temperature steam, in a manner in which the water content of the fibers materials does not exceed around 25% by weight, where the high temperature steam can be saturated steam, slightly superheated steam, or superheated steam, and, optionally,
b) subjecting the steam-treated fibers to a disinfecting treatment, wherein the disinfecting treatment is selected from the group consisting of UV light, ethylene oxide, methyl bromide, and supercritical or subcritical carbon dioxide, under conditions sufficient to remove any microbial contaminants that may be present.

19. A process for disinfecting, and, optionally, sterilizing, fibers, comprising:
a) subjecting fibers to high temperature steam, in a manner in which the water content of the fibers does not exceed around 25% by weight, where the high temperature steam can be saturated steam, slightly superheated steam, or superheated steam, and
b) subjecting the steam-treated non-woven materials to a disinfecting treatment, wherein the disinfecting treatment comprises treating the materials with a sufficient amount of ozone to remove any microbial contaminants that may be present.

20. The process of claim 18 or 19, wherein the water content of the fibers does not exceed around 15% by weight.

21. The process of claim 18 or 19, wherein the disinfecting treatment is UV light.

22. The process of claim 21, wherein the UV light is UV-C radiation at a wavelength between 100 nanometers and 280 nanometers.

23. The process of claim 18 or 19, wherein, before or after being subjected to high-temperature steam, one or more chemicals are applied to the fibers,
wherein the chemicals are selected from the group consisting of pesticides, insecticides, fertilizers, antimicrobials, antivirals, antimycotics, antibacterials, antirickettsials, antibiotics, biocides, biostats, and mixtures thereof.

24. The process of claim 23, wherein the antimicrobials are quaternary ammonium salts.

25. The process of claim 18 or 19, wherein the fibers comprise virgin fibers, fibers obtained by deconstructing post-consumer and/or post-industrial waste, or combinations thereof.

26. The process of claim 25, wherein the post-consumer and/or post-industrial waste comprises used textiles and/or leather materials.

27. The process of claim 15, wherein combinations of fibers are used, and wherein the combinations of fibers are subjected to an intimate mixing step to provide homogeneity to the fibers, such that the composition of the blend is fairly uniform, wherein fairly uniform means that the composition varies by no more than 20% in any portion of the blended fibers.

28. The process of claim 18 or 19, wherein the fibers are applied to an air card.

29. The process of claim 28, wherein the fibers are subjected to one or more carding steps.

30. The process of claim 29, wherein the carded fibers are subjected to hydroentanglement or needlepunching steps to form a non-woven material.

31. The process of claim 30, wherein the non-woven material is subjected to a calendaring step.

32. The process of claim 18 or 19, wherein the fibers are subjected to thermal, chemical, or adhesive bonding processes to form a non-woven material.

33. The process of any of claims 30 to 32, wherein one or more chemicals are applied to the non-woven material,
wherein the chemicals are selected from the group consisting of pesticides, insecticides, fertilizers, antimicrobials, antivirals, antimycotics, antibacterials, antirickettsials, antibiotics, biocides, biostats, and mixtures thereof.

34. The process of claim 33, wherein the antimicrobials are quaternary ammonium salts.

35. The process of any of claims 30 to 32, further comprising subjecting the non-woven materials to high-temperature steam and a disinfecting treatment,
wherein the high temperature steam can be saturated steam, slightly superheated steam, or superheated steam, and the disinfecting treatment is selected from the group consisting of UV light, ethylene oxide, methyl bromide, and supercritical or subcritical carbon dioxide, under conditions sufficient to remove any microbial contaminants that may be present.

36. The process of claim 35, wherein the disinfecting treatment is UV light.

37. The process of claim 36, wherein the UV light is UV-C radiation at a wavelength between 100 nanometers and 280 nanometers.

38. The process of any of claims 30 to 32, further comprising subjecting the non-woven materials to a disinfecting process comprising treatment with ozone.

39. The process of any of claims 30 to 32, wherein the non-woven material is formed into sheets for use in personal care, baby care, cosmetic applications, household cleaning, automotive applications, or industrial cleaning applications.

40. A device for preparing disinfected non-woven materials from fibers, comprising:
a blend line for blending fibers,
a blend line storage box for storing the blended fibers,
a first conveyor belt to convey the blended fibers,
a non-woven card, to align the fibers,
a hydroentanglement or needlepunching system comprising a dewatering belt,
an optional chemical dosing system to apply a chemical treatment to the non-woven material,
a drier,
a calender,
a module that applies high temperature steam to the non-woven material, which can be saturated steam, slightly superheated steam, or superheated steam, which module comprises one or a plurality of nozzles through which the high temperature steam can be applied,
a module which applies UV light in a sufficient amount and for a sufficient duration of time to effectively destroy any residual contamination on the non-woven material, and
one or more conveyor belts to convey the fibers and/or non-woven materials through the apparatus.

41. The apparatus of claim 40, further comprising an airlaid card to receive the blended fibers from the storage box.

42. The apparatus of claim 40, further comprising a winder for winding up the non-woven material.
